(19) United States
(12) Patent Application Publication
(10) Pub. No.: US 2018/0073081 A1
(43) Pub. Date: Mar. 15, 2018

(54) Method for Predicting Prognostic Results of Diseases

(71) Applicant: National Taiwan University, Taipei (TW)

(72) Inventors: Eric Y. Chuang, Taipei City (TW); Mong-Hsun Tsai, Taipei City (TW); Wen-Chien Chou, Taipei City (TW); Liang-Chuan Lai, Taipei City (TW); Hwei-Fang Tien, Taipei City (TW); Yu-Chiao Chiu, Taipei City (TW); Tzu-Pin Lu, Taipei City (TW); Yen-Chun Liu, Taipei City (TW); Yi-Yi Kuo, Taipei City (TW); Hsin-An Hou, Taipei City (TW); Yidong Chen, San Antonio, TX (US)

(21) Appl. No.: 15/263,758

(22) Filed: Sep. 13, 2016

(51) Int. Cl. C12Q 1/68; G06F 19/00

(52) U.S. Cl. CPC C12Q 1/6886; G06F 19/3431; C12Q 2600/156; C12Q 2600/178; C12Q 2600/158; C12Q 2600/118

(57) ABSTRACT

The invention is directed to a method to predict prognostic results for diseases including acute myeloid leukemia (AML) by analyzing novel markers which comprises microRNA/mRNA (miRNA/mRNA) pairings. In particular, the miRNA/mRNA pairings are a kind of NPM1 mutation-modulated miRNA/mRNA regulation pairs.

### BACKGROUND OF THE INVENTION

### 1. Field of the Invention

The invention is directed to a method for predicting prognostic results of diseases including acute myeloid leukemia (AML) by analyzing novel markers which comprises microRNA/mRNA (miRNA/mRNA) pairings. In particular, the miRNA/mRNA pairings are a kind of NPM1 mutation-modulated miRNA/mRNA regulation pairs.

### 2. Description of the Prior Art

Acute myeloid leukemia (AML) arises from a sequence of genetic mutations, among which alterations of the nucleophosmin (NPM1) gene are frequent, being present in about one third of adult AML patients and up to 50% of those with a normal-karyotype. NPM1 is a multifunctional protein with both tumor suppressor and oncogene functions and is associated with cell cycle progression, response to oncogenic stimuli, and apoptosis. Studies have shown the prognostic significance of NPM1 mutation in AML and its close association with other gene mutations. Distinct microRNA (miRNA), mRNA, and miRNA-mRNA signatures have been reported in NPM1-mutated AML cells. However, it has not been explored yet whether the mutation participates in the complex interaction between miRNA and mRNA, miRNAs, a group of short non-coding RNAs, participate in gene regulation by complementary binding to the 3′ untranslated regions of target mRNAs. They are estimated to target and suppress over one third of human genes, and their aberrant expression is associated with leukemogenesis and prognosis in AML patients. Some miRNA/mRNA pairs have been validated through in vitro experiments; however, most of the validation experiments were performed under a restricted condition in a particular cell line. Since biological systems are substantially dynamic, there would be considerable variations in the regulatory strength of miRNA/mRNA pairings, especially in cancer cells. Dynamic regulation of miRNA-mRNAs is reported to harbor prognostic significance in glioblastoma and breast cancer, while its involvement in AML, as well as its association with NPM1 mutation, remains an uncharacterized territory.

### SUMMARY OF THE INVENTION

In the present invention, firstly we disclose a method for predicting prognostic results of diseases including acute myeloid leukemia. The method comprises analyzing regulatory strengths of a NPM1 mutation-modulated miRNA/mRNA regulation (MMR) pair in a subject. The miRNA/mRNA regulation (MMR) is dynamic in AML, and the dynamicity could be modulated by NPM1 mutation. We systematically analyzed sample-matched mRNA and miRNA microarray datasets derived from a discovery cohort of 181 de novo AML patients and identified hundreds of NPM1 mutation-modulated MMR pairs. We validated the NPM1 mutation-modulated regulation using three approaches, including (1) in silico validation in two independent cohorts, (2) a high-throughput dataset derived from OCI/AML3 cell line (which harbors endogenous heterozygous NPM1 mutation) with the NPM1 mutation specifically knocked down by siRNA, and (3) comparison of the expression levels of mRNA targets of selected miRNAs between cells with different burdens of NPM1 mutation. We also discussed the functional effects of such differential regulation and its prognostic significance in predicting overall survival (OS) in AML patients. Overall, this invention illustrates a prognostic and regulatory layer of miRNA/mRNA interactions that could be modulated by NPM1 mutation.

In one embodiment, the NPM1 mutation-modulated miRNA/mRNA regulation (MMR) pair comprises hsa-miR-125b, hsa-miR-193b, hsa-miR-320 and hsa-miR-376c.

In one embodiment, the NPM1 mutation-modulated miRNA/mRNA regulation (MMR) pair comprises PCTP, GMCL1, KIAA0182, ARID4B, MEF2C, ABCC4, ANKRD10, BIN2 and ST6GAL1.

In one embodiment, the NPM1 mutation-modulated miRNA/mRNA regulation (MMR) pair is selected from one of the group consisting of hsa-miR-125b/PCTP, hsa-miR-125b/ABCC4, hsa-miR-125b/BIN2 and hsa-miR-125b/ST6GAL1.

In another embodiment, the NPM1 mutation-modulated miRNA/mRNA regulation (MMR) pair is selected from one of the group consisting of hsa-miR-193b/KIAA0182 and hsa-miR-193b/ARID4B.

In a certain embodiment, the NPM1 mutation-modulated miRNA/mRNA regulation (MMR) pair is selected from one of the group consisting of hsa-miR-320/GMCL1 and hsa-miR-320/ANKRD10.

In a certain embodiment, the NPM1 mutation-modulated miRNA/mRNA regulation (MMR) pair is hsa-miR-376c/MEF2C.

In a certain embodiment, the subject comprises cell samples, tissue sections, blood sample and lymph sample. Preferably, the cell samples comprise OCI/AML3 and NPM1-WT K562.

In another objective of the present invention, we also disclose a prognostic scoring system. The scoring system is constructed as the following equation:

Score=0.36×C_{miR-125b/PCTP}+0.25×C_{miR-320/GMCL1}+0.21×C_{miR-193b/KIAA0182}+0.23×C_{miR-193b/ARID4B}+0.24×C_{miR-376c/MEF2C}+0.02×C_{miR-125b/ABCC4}+0.11×C_{miR-320/ANKRD10}+0.03×C_{miR-125b/BIN2}+0.19×C_{miR-125b/ST6GAL1},

where C denotes the covariability of a miRNA and its modulated target mRNA.

In an embodiment, the prognostic scoring system is applied to predict acute myeloid leukemia (AML) prognosis

### BRIEF DESCRIPTION OF THE DRAWINGS

FIGS. 1(a) to 1(b) illustrate dynamic MMR in AML. Herein, FIG. 1.(a) is histogram of pairwise correlation coefficients for 798 biologically (in vitro) validated miRNA-mRNA pairs in the NTUH discovery dataset (n=181). The dashed and solid lines denote zero and the cutoff for significant negative correlation (one-tailed P<0.01) for a sample size of 181, respectively. The percentage shows the portion of significantly negatively correlated pairs; FIG. 1.(b) is illustration of NPM1 mutation-modulated MMR. In this study we hypothesized that the strength of negative regulation of an miRNA on its target mRNA can be attenuated by mutation status of the NPM1 gene;

FIGS. 2(a) to 2(c) illustrate NPM1 mutation-modulated MMR pairs and network, and the associated functions. Herein, FIG. 2. (a) illustrates scatter plots of the most significant NPM1 mutation-modulated MMR pair, hsa-miR-193b/NRIP1. Negative regulation between hsa-miR-193b and NRIP1 developed in NPM1-WT samples, but the negative regulation was lost in NPM1-MUT samples (P of differential correlation=3.1×10₋₁₁). FIG. 2.(b) shows that we merged 493 such modulated MMR pairs revealed by a systematic analysis into the NPM1 mutation-modulated MMR network. Filled and empty nodes denote miRNAs and target mRNAs, respectively; FIG. 2.(c) illustrates functional categories of miRNA-mRNA pairs in the regulatory network revealed by Ingenuity Pathway Analysis. The height of bars denotes the significance levels of functions and diseases assessed by the Fisher's exact test;

FIGS. 3(a) to 3(b) illustrate In silico validation of NPM1 mutation-modulated MMR by independent AML cohorts. Herein, FIG. 3. (a) is enrichment plots of the 493 miRNA-mRNA pairs identified from the discovery cohort in two validation datasets (TCGA validation and NTUH validation) generated by a gene set-based analytical software GSEA. GSEA ranked each of the 35,542 putative miRNA-mRNA pairs along the horizontal axis based on their differences in normalized correlation coefficients between NPM1-MUT and NPM1-WT states (indicated by left and right arrows, respectively) in a validation dataset. GSEA adopted a running sum method (the curve) to calculate an enrichment score for measuring the degree to which the 493 miRNA-mRNA pairs (denoted as line segments) are overrepresented, or enriched, at the NPM1-WT side of the ranked list. A negative enrichment score represents an overall trend of NPM1-WT-specific negative regulation. Significance of the enrichment score was assessed by a permutation test. As a result, the identified 493 miRNA-mRNA pairs showed significant NPM1-WT-specific regulation in both TCGA and NTUH validation datasets. FIG. 3. (b) is comparison of leading-edge subsets of the two validation datasets. GSEA identified a leading-edge subset as the core of miRNA-mRNA pairs (denoted by a dashed circle in (a)) that accounted for the significant enrichment score. Significance of overlap was assessed by chi-square test;

FIGS. 4(a) to (e) illustrate In vitro validation of NPM1 mutation-modulated MMR by the OCI/AML3 cell model. Herein, FIG. 4. (a) shows two experimental design of the OCI/AML3 cell line model. Left panel, for systematically validating all identified NPM1 mutation-modulated MMR, an in vitro model was constructed by electroporation of NPM1 mutation (mNPM1)-specific or scramble siRNA into OCI/AML3 cells to mimic NPM1-WT (OCI/AML3-shNPM1mut) or NPM1-MUT (OCI/AML3-scramble) conditions, respectively, followed by microarray profiling of all miRNAs and mRNAs (results in (d)). Right panel, to further investigate a set of identified MMR pairs, another model was built by electroporation of the siRNA (or scramble control) together with mirVana miRNA mimic (or control), followed by quantification of modulated target mRNAs of the miRNA (results in (e)). FIG. 4. (b) is confirmation of repression efficiency of the siRNA in the expression level (RT-qPCR) and FIG. 4.(c) shows protein level (Western blots and quantitated values) in OCI/AML3 cells. Abbreviation: mNPM, mutated nucleophosmin FIG. 4. (d) is systematic validation of all 493 NPM1 mutation-modulated MMR in OCI/AML3 cells. We generated the cumulative distribution curves of differences in correlation coefficients of the 493 MMR pairs between OCI/AML3-shNPM1mut (n=3) and OCI/AML3-scramble (n=3) cells. A left shifted curve represents overall enhanced regulatory strengths in the NPM1-WTmimicking context. Statistical significance was assessed by the one-sample t-test (against zero) and Kolmogorov-Smirnov test (against the standard normal distribution). FIG. 4.(e) is validation of the selected modulated target mRNAs of hsa-miR-320, hsa-miR-145, and hsa-let-7c in OCI/AML3 cells. Expression of the mRNAs was profiled using RT-qPCR. The vertical axis denotes the relative fold changes in mRNA expression upon miRNA overexpression between OCI/AML3-shNPM1mut and OCI/AML3-scramble cells; i.e., ratios less than 1 represent enhanced regulation in OCI/AML3-shNPM1mut (i.e. NPM1-WT-like) cells. Underlined are mRNAs showing enhanced down-regulation (median ratio <1) upon miRNA transfection in the NPM1-WT-like condition. Horizontal lines in the body and whiskers of the box plot represent quartiles and extreme values, respectively. P-values from one-sample t-test against unity are symbolized as t, trend of change (P<0.15);*, P<0.05; **, P<0.01; ***, P<0.001;

FIGS. 5(a) to 5(c) illustrate In vitro validation of NPM1 mutation-modulated MMR by the K562 cell model. Herein, FIG. 5.(a) is experimental design of the K562 cell line model. The in vitro model was constructed by electroporation of control and NPM1 mutation-expressing constructs, to generate K562-control (i.e. NPM1-WT) and K562-NPM1mut (i.e. NPM1-MUT-like) conditions, respectively, together with mirVana miRNA mimic (or control), followed by quantification of modulated target mRNAs of the miRNA. FIG. 5.(b) is confirmation of the mutated NPM1-expressing construct in the expression level (left panel) and protein level (right panel). Abbreviation: N.D., not detected. FIG. 5.(c) is validation of the selected modulated target mRNAs of hsa-miR-320, hsa-miR-145, and hsa-let-7c in K562 cells. Expression of the mRNAs was profiled using RT-qPCR. PLXNC1 and GAS7 were excluded for their low endogenous expression in K562 cells. The vertical axis denotes ratios of relative changes in mRNA expression upon miRNA overexpression between K562-control and K562-NPM1mut cells. Underlined are mRNAs showing enhanced down-regulation upon miRNA transfection in the K562-control cells (i.e. the NPM1-WT condition). Statistical significance from one-sample t-test against unity are symbolized as t, trend of change (P<0.15);*, P<0.05; **, P<0.01; and

FIGS. 6(a) to 6(d) illustrate prognostic significance of NPM1 mutation-modulated MMR. Herein, FIG. 6.(a) is Kaplan-Meier curves of covariability of hsa-miR-125b and its modulated target PCTP in the NTUH discovery dataset. Covariability measures the magnitude of inverse changes in the expression levels of a miRNA and its target mRNA (i.e., the regulatory strength). Patients with stronger regulation (higher covariability) between hsa-miR-125b and PCTP had significantly better OS. FIG. 6.(b) is validation of the prognostic value of hsa-miR-125b/PCTP in the TCGA dataset. Detailed results of multivariable analysis in both datasets are provided in Table 1. FIG. 6.(c) is Kaplan-Meier curves of the prognostic scoring system in the NTUH discovery dataset. The score was calculated by a linear combination (weighted sum) of the 9 prognostic MMR pairs. FIG. 6. (d) is Kaplan-Meier curves of the prognostic score in the TCGA dataset. Results of multivariable analysis in both datasets are provided in Table 2.

### DETAILED DESCRIPTION OF THE PREFERRED EMBODIMENTS

In a first embodiment, the claimed invention provide a method for predicting prognostic results of diseases including acute myeloid leukemia. The method comprises analyzing regulatory strengths of a NPM1 mutation-modulated miRNA-mRNA regulation (MMR) pair in a subject. The miRNA-mRNA regulation (MMR) is dynamic in AML, and the dynamicity could be modulated by NPM1 mutation.

Three approaches are performed for validating the NPM1 mutation-modulated regulation, including (1) in silico validation in two independent cohorts, (2) a high-throughput dataset derived from OCI/AML3 cell line (which harbors endogenous heterozygous NPM1 mutation) with the NPM1 mutation specifically knocked down by siRNA, and (3) comparison of the expression levels of mRNA targets of selected miRNAs between cells with different burdens of NPM1 mutation.

In one example of the first embodiment, the NPM1 mutation-modulated miRNA/mRNA regulation (MMR) pair comprises hsa-miR-125b, hsa-miR-193b, hsa-miR-320 and hsa-miR-376c.

In one example of the first embodiment, NPM1 mutation-modulated miRNA/mRNA regulation (MMR) pair comprises PCTP, GMCL1, KIAA0182, ARID4B, MEF2C, ABCC4, ANKRD10, BIN2 and ST6GAL1.

In one preferred example of the first embodiment, the NPM1 mutation-modulated miRNA/mRNA regulation (MMR) pair is selected from one of the group consisting of hsa-miR-125b/PCTP, hsa-miR-125b/ABCC4, hsa-miR-125b/BIN2 and hsa-miR-125b/ST6GAL1.

In one preferred example of the first embodiment, the NPM1 mutation-modulated miRNA/mRNA regulation (MMR) pair is selected from one of the group consisting of hsa-miR-193b/KIAA0182 and hsa-miR-193b/ARID4B.

In a certain example of the first embodiment, the NPM1 mutation-modulated miRNA/mRNA regulation (MMR) pair is selected from one of the group consisting of hsa-miR-320/GMCL1 and hsa-miR-320/ANKRD10.

In a certain example of the first embodiment, the NPM1 mutation-modulated miRNA/mRNA regulation (MMR) pair is hsa-miR-376c/MEF2C.

In a certain example of the first embodiment, the subject comprises cell samples, tissue sections blood samples and lymph samples. Preferably, the cell samples comprise OCI/AML3 and NPM1-WT K562.

In another embodiment, the claimed invention also discloses a prognostic scoring system. The scoring system is constructed as the following equation:

Score=0.36×C_{miR-125b/PCTP}+0.25×C_{miR-320/GMCL1}+0.21×C_{miR-193b/KIAA0182}+0.23×C_{miR-193b/ARID4B}+0.24×C_{miR-376c/MEF2C}+0.02×C_{miR-125b/ABCC4}+0.11×C_{miR-320/ANKRD10}+0.03×C_{miR-125b/BIN2}+0.19×C_{miR-125b/ST6GAL1},

where C denotes the covariability of a miRNA and its modulated target mRNA.

In a preferred example of another embodiment, the prognostic scoring system is applied to predict acute myeloid leukemia (AML) prognosis.

Materials and Methods for Evaluating miRNA and mRNA Expression Datasets of AML Patients

We analyzed sample-paired miRNA and mRNA expression profiles from three independent AML cohorts, one as discovery and two for validation. The discovery dataset was derived from bone marrow samples of 181 de novo AML patients, 136 NPM1-wild type (NPM1-WT) and 45 NPM1-mutated (NPM1-MUT), enrolled at the National Taiwan University Hospital (NTUH). These patients were simultaneously analyzed for miRNA expression using TaqMan Array Human MicroRNA A Cards v2.0 (Applied Biosystems, Foster City, Calif.) and mRNA profiles using Human HT-12 v4.0 BeadChips (Illumina, San Diego, Calif.). One of the validation datasets was the 184-sample (140 NPM1-WT and 44 NPM1-MUT) dataset of AML cohort from The Cancer Genome Atlas (TCGA).₁₄ The patients were profiled for miRNA and mRNA expression by Genome Analyzer miRNA Sequencing (Illumina, San Diego, Calif.) and Human Genome U133 Plus 2.0 Array (Affymetrix, Santa Clara, Calif.), respectively. The other validation dataset was profiled from 109 AML patients (98 NPM1-WT and 11 NPM1-MUT) of NTUH, of which miRNA and mRNA expression was analyzed by nCounter miRNA Expression Assays (NanoString, Seattle, Wash.) and Human HT-12 v4.0 BeadChips (Illumina, San Diego, Calif.), respectively; the datasets have been deposited in the Gene Expression Omnibus database (accession number GSE68469). This study was performed in accordance with the Declaration of Helsinki and was approved by the Research Ethics Committee of NTUH.

Systematic Identification of NPM1 Mutation-Modulated MMR Pairs

We developed a statistical framework to systematically screen the ‘putative miRNA/mRNA targeting pairs’ for NPM1 mutation-modulated MMR pairs, in which regulatory strength (measured by Pearson correlation coefficient (r)) shows a significant attenuation in NPM1-MUT patients compared to NPM1-WT patients. Here a total of 35,542 putative miRNA/mRNA targeting pairs was studied, including 798 previously biologically (in vitro) validated pairs and 34,744 predicted in silico by at least 2 of the 3 prediction algorithm, (data downloaded from the miRSystem database). Mathematically, an NPM1 mutation-modulated MMR pair satisfies all the following criteria:

i) With significant negative r in NPM1-WT (correlation P<0.01).
ii) Without significant negative r in NPM1-MUT (correlation P>0.1).
iii) With significant change in absolute r between NPM1-WT and NPM1-MUT, i.e., significant |r_{NPM1-WT}|−|r_{NPM1-MUT}|, where the correlation coefficients were normalized to eliminate the biases generated from different sample sizes. Statistical significance of such changes was assessed by a simulation test (P<0.01).

Functional Annotation Analysis and Gene Set Enrichment Analysis

We performed functional annotation analysis by using the knowledge-based Ingenuity Pathway Analysis (Qiagen, Redwood City, Calif.) software. The Gene Set Enrichment Analysis (GSEA)software was used in our validation analysis to test whether a set of miRNA/mRNA pairs (identified from the discovery dataset) showed an overall enrichment (i.e., over-representation) in NPM1-WT samples, compared to NPM1-MUT samples, in a validation cohort. Statistical significance of the degree of enrichment was assessed using a 2,000-time random permutation test.

Survival Analysis Based on Covariability of MMR Pairs

We employed the covariability measure to model the regulatory strength of an miRNA/mRNA pair for each patient. Conceptually, covariability is a per-sample analog to Pearson correlation coefficient r. It measures the magnitude of changes in an miRNA and its target mRNA in the opposite direction in one sample; i.e., the larger the covariability is, the stronger the negative regulation is between an miRNA and an mRNA. We utilized Cox univariable and multivariable proportional hazards regression models to analyze OS of patients with one and multiple factors, respectively. We used Kaplan-Meier curve and log-rank test to compare OS of two groups of patients (e.g., high covariability vs. low, or high prognostic score vs. low). To eliminate possible statistical biases arising from samples around the borderline of two groups, we conducted the Kaplan-Meier curve and log-rank test between patients with high (within the fourth quartile) and low (first quartile) covariability (or score), while the Cox regression, as a continuous model, was conducted based on all patients.

Cell Lines

The human AML cell line OCI/AML3 carrying endogenous heterozygous NPM1mutation and the NPM1-WT leukemia cell line K562 were used for in vitro validation tests.

Knockdown of NPM1 Mutant in OCI/AML3 Cells by NPM1 Mutation-Specific siRNA

The mutated allele of NPM1 in OCI/AML3 cells was knocked down by electroporation using the siRNA (Thermo Fisher Scientific, Wilmington, Del.) that specifically targets the mutated NPM1 allele but not the wild-type allele to generate OCI/AML3-shNPM1mut cells, mimicking wild-type NPM1 condition. The scramble siRNA was electroporated into OCI/AML3 cells to generate OCI/AML3-scramble cells (NPM1-mutated condition) as a control. Cells were washed with PBS and re-suspended with Buffer R (Life Technologies, Grand Island, N.Y.) before electroporation. The electroporation was performed at 1100 V, 20 msec., and 3 pulses using the Neon transfection system (Life Technologies, Grand Island, N.Y.).

Expression of Mutant NPM1 in K562 Cells Using Expression Construct

We generated an expression construct of NPM1 mutation by cloning mutated NPM1 into the pCDH-CMV-MCS-EF1-Puro vector (SBI, Mountain View, Calif.), and then transfected it into K562 cells by electroporation to generate K562-NPM1mut cells. As a control, the empty vector was transfected into K562 cells to form NPM1-WT K562-control cells. Cells were re-suspended with BTXpress High Performance Electroporation Solution (BTX, Holliston, Mass.) before electroporation. The electroporation was performed at 160V, 950 ρF, and no resistance using a BTX ECM 630 electroporator (BTX, Holliston, Mass.).

miRNA and mRNA Microarray Profiling of OCI/AML3 Cells

RNA samples from OCI/AML3-shNPM1mut and OCI/AML3-scramble cells were prepared from three biological replicates in both cells and hybridized to nCounter miRNA Expression Assays (NanoString, Seattle, Wash.) and Human HT-12 v4.0 BeadChips for miRNA and mRNA expression profiling, respectively, according to the manufacturer's instructions.

miRNA Overexpression in Cells

To further see if the differential regulation of selected target miRNA-mRNA pairs was modulated by NPM1 mutation, mirVana control or mirVana miRNA mimics (Life Technologies, Grand Island, N.Y.) of selected miRNAs was electroporated into OCI/AML3-shNPM1mut and OCI/AML3-scramble cells (or K562-control and K562-NPM1mut cells), followed by RT-qPCR of selected NPM1 mutation-modulated target mRNAs.

RT-qPCR and Immunoblotting Analyses for In Vitro Studies

ΔCₜ values (cycle numbers with respect to an internal control) of selected modulated mRNAs from RT-qPCR assay were compared between samples transfected with miRNA mimics and controls to achieve ΔΔCₜ. We then compared the ΔΔCₜ values between OCI/AML3-shNPM1mut and OCI/AML3-scramble (or K562-control vs. K562-NPM1mut) cells. Western blotting analysis was performed by using our customized rabbit polyclonal antibody (CA544), which specifically recognizes the mutated nucleophosmin. The epitope of the antibody is LCLAVEEVSLRK (the mutated peptide sequence in type A NPM1 mutation).

Dynamic MMR in AML

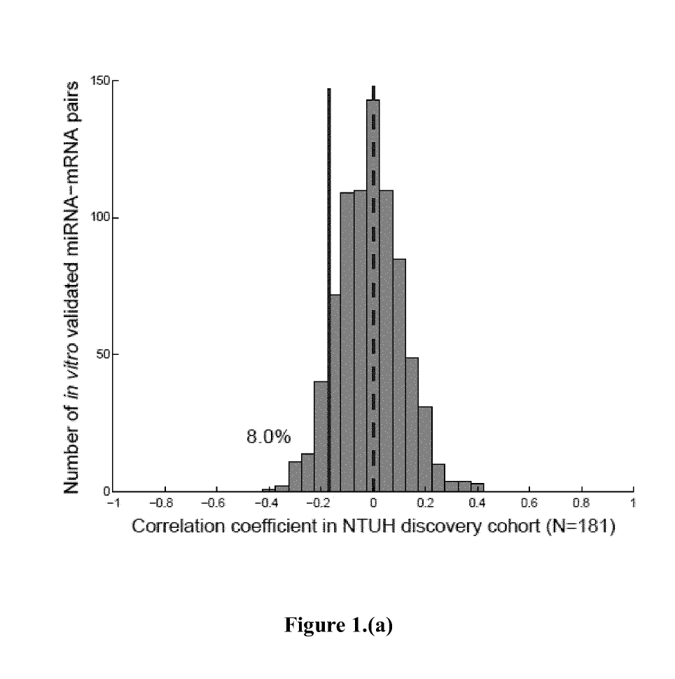

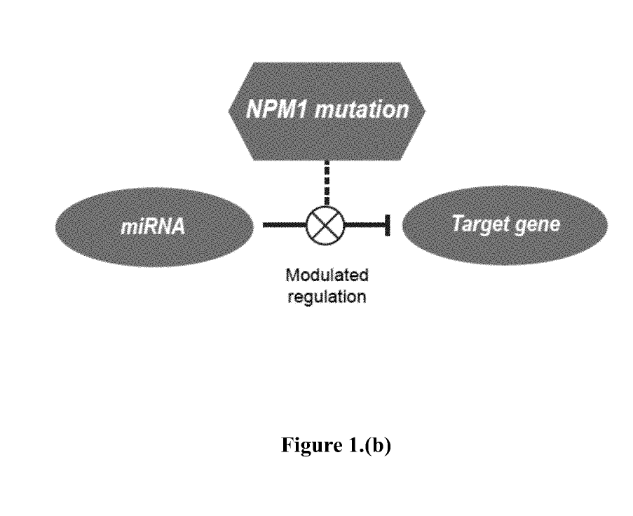

To explore the dynamic miRNA-mRNA regulatory relationship in AML, we investigated the sample-paired mRNA and miRNA microarray datasets of 181 AML patients recruited at NTUH (the discovery dataset; see Materials and Methods). With a systematic analysis of the paired 181 microarray profiles, we found that the expected negative pairwise correlation coefficients (r) were not observed among the 798 previously biologically validated MMR pairs. Instead, r was nearly randomly distributed (FIG. 1a), and only 8.0% (64 out of 798) showed significant negative correlation in our analysis (with one-tailed P of r<0.01; FIG. 1a) Similar results were identified in two validation datasets, namely the TCGA validation (n=184) and NTUH validation (n=109). The data suggest that regulatory strength between miRNAs and mRNAs is not maintained constantly across all AML patients. Furthermore, the expected negative regulation in the 35,542 putative miRNA-mRNA pairs predicted in silico by miRNA-target prediction algorithms was considerably attenuated in NPM1-MUT patients compared to NPM1-WT patients. Taken together, these observations suggest that MMR is dynamic and could be modulated by NPM1 mutation (illustrated in FIG. 1b).

Identification of NPM1 Mutation-Modulated MMR Pairs

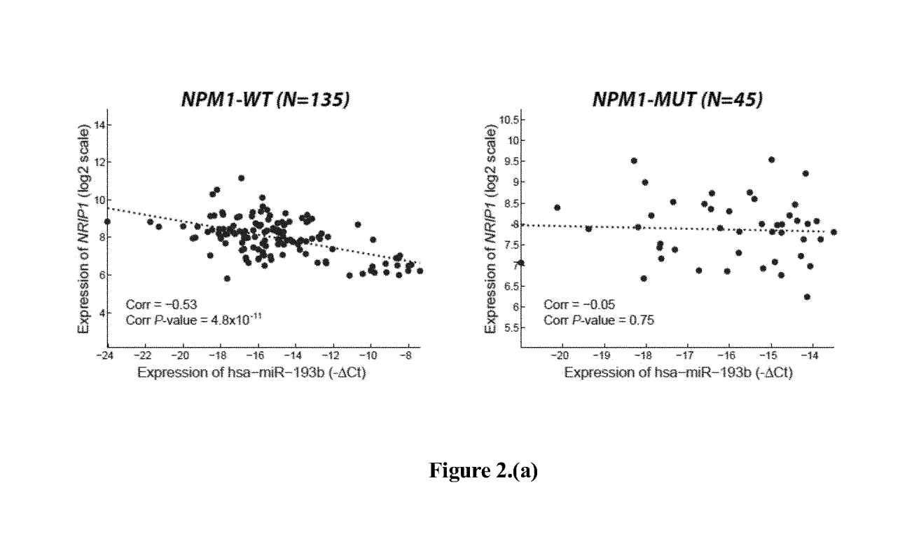

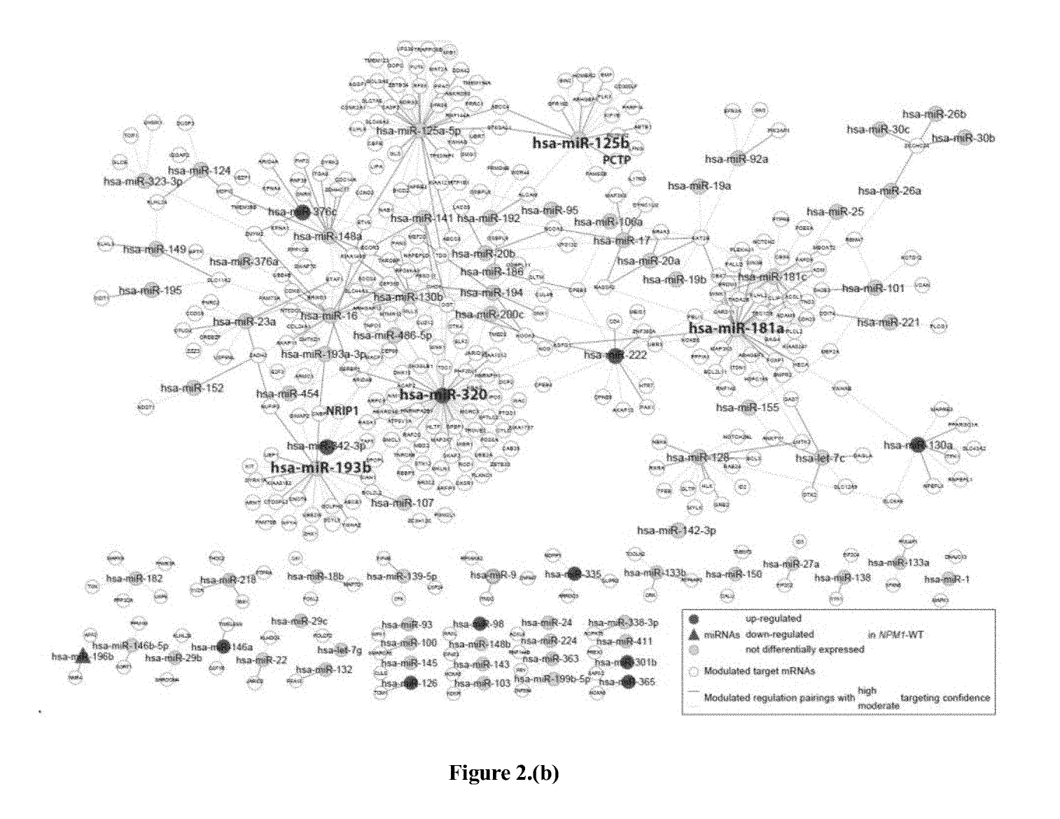

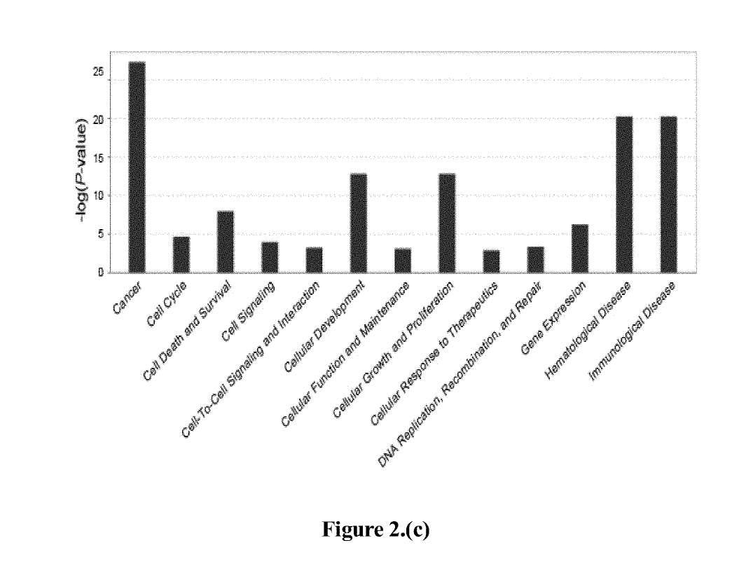

We sought to systematically identify the MMR pairs in which the strength of regulation is modulated by NPM1 mutation, i.e., a more obvious regulatory relationship is seen in NPM1-WT patients compared to NPM1-MUT patients. We developed a Pearson correlation-based framework to infer the statistical significance of changes in regulatory strength associated with NPM1 mutation. By screening the 35,542 putative miRNA-mRNA pairs, we identified 493 MMR pairs exhibiting NPM1-WT-specific regulation. These NPM1 mutation-modulated pairs involved 84 miRNAs and 363 mRNAs. The most significant one was hsa-miR-193b/NRIP1 (P of differential correlation=3.1×10₋₁₁; FIG. 2a), which was strongly negatively correlated (r=−0.53, P=4.8×10₋₁₁) in NPM1-WT but almost uncorrelated in NPM1-MUT (r=−0.05, P=0.75). We noted that differential miRNA or mRNA expression levels between NPM1-WT and NPM1-MUT could not explain the NPM1 mutation modulation; in fact, only 14.3% and 5.5% of the miRNAs and mRNAs, respectively, were differentially expressed between the NPM1-WT and NPM1-MUT patients (with Bonferroni-corrected t-test P<0.05). These data indicated that NPM1 mutation-modulated MMR was through a mechanism other than direct regulation of expression levels. To further explore this observation, we merged the 493 MMR pairs to construct an NPM1 mutation-modulated MMR network by the open source software Cytoscape (FIG. 2b). On average, each miRNA regulated ˜4.3 mRNAs in the NPM1-WT-specific context. hsa-miR-320 and hsa-miR-181a, previously reported to be associated with AML prognosis,₃₀ were the two most prominent hub miRNAs in the network, with connections to 62 and 42 mRNAs, respectively (FIG. 2b), suggesting their crucial roles in signaling modulated by NPM1 mutation.

Functions of NPM1 Mutation-Modulated MMR

Functional analysis of NPM1 mutation-modulated MMR illustrated the enrichment of the network in biological functions associated with cancer and hematological diseases, as well as those essential for AML, such as cell cycle, cell death and survival, and cellular response to therapeutics (P-values<0.01; FIG. 2c). Twenty-two miRNAs and mRNAs were associated with acute leukemia, including KIT, KRAS, hsa-miR-155, hsa-miR-181a, and hsa-miR-320. Several known functions of NPM1 mutation, such as cell death and proliferation of leukemia cell lines. The results suggest that NPM1 mutation performs its functions, at least partially, through modulating MMR.

In Silico Validation Analysis in Independent AML Cohorts

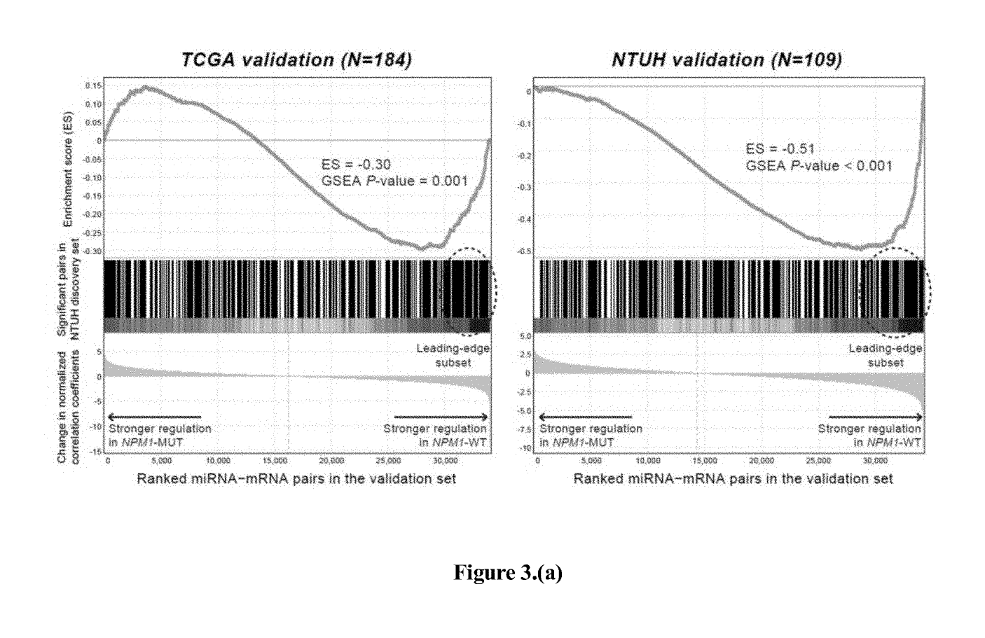

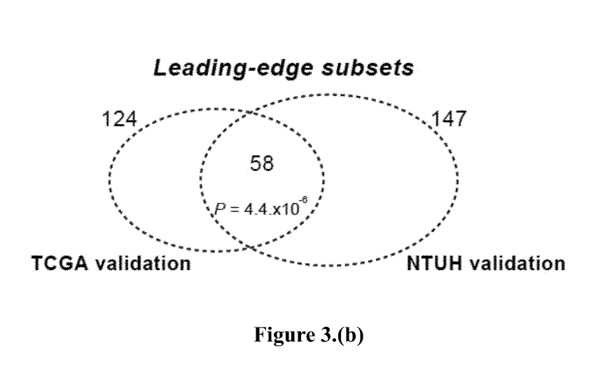

We tested whether the 493 NPM1 mutation-modulated MMR pairs showed concordant enhanced negative regulation in NPM1-WT compared to NPM1-MUT in two validation cohort datasets. Remarkably, the 493 MMR pairs exhibited significant NPM1-WTspecific regulation in both validation datasets as shown by GSEA (P=0.001 and P<0.001, respectively; FIG. 3a-b). Analysis of the core MMR pairs revealed by GSEA (i.e., the most significant pairs in each dataset; denoted in FIG. 3a-b) showed significant overlap between the two datasets (chi-square test P=4.4×10₋₆; FIG. 3c), suggesting the consistency of the modulated regulation. Furthermore, NPM1 mutation-modulated MMR pairs independently identified from the two validation datasets were significantly concordant with those from the NTUH discovery dataset (chi-square test P=1.8×10₋₁₃ and ˜0, respectively). It is noteworthy that the validation datasets were derived from different platforms of microarrays and next-generation sequencing. Taken together, the data suggest the consistency of NPM1 modulation in MMR across cohorts and profiling techniques.

In Vitro Validation Studies by Two Cell Line Models

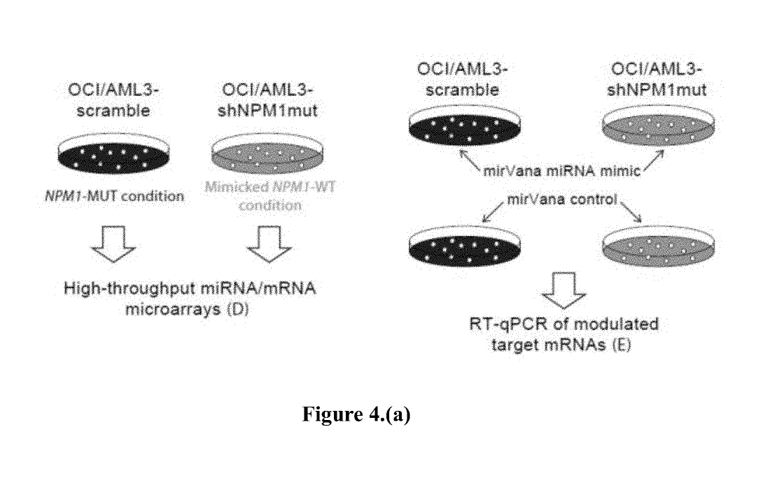

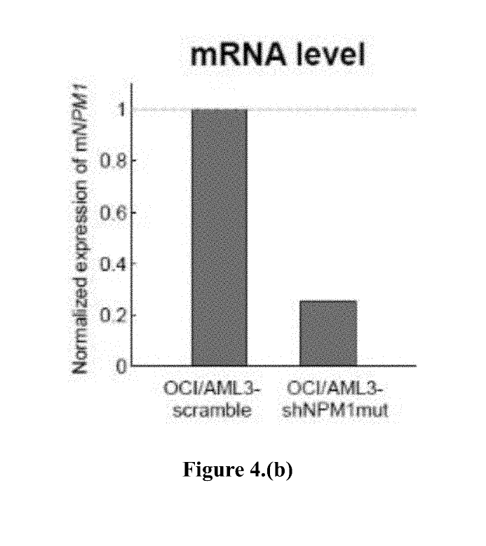

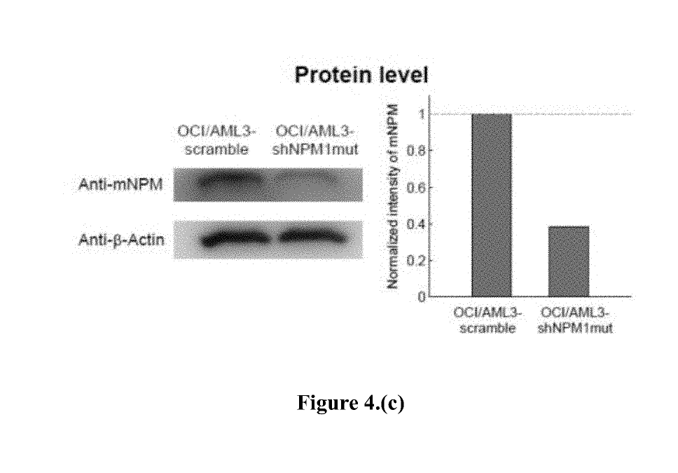

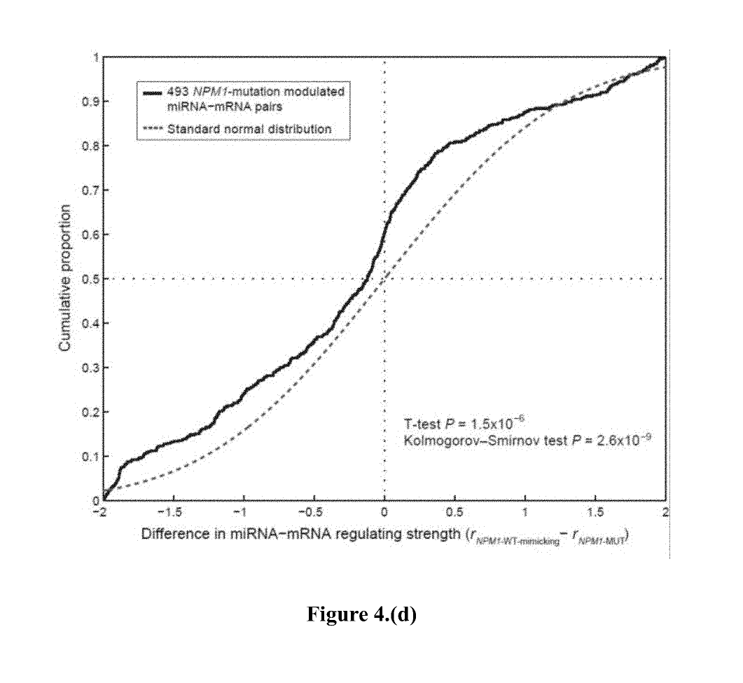

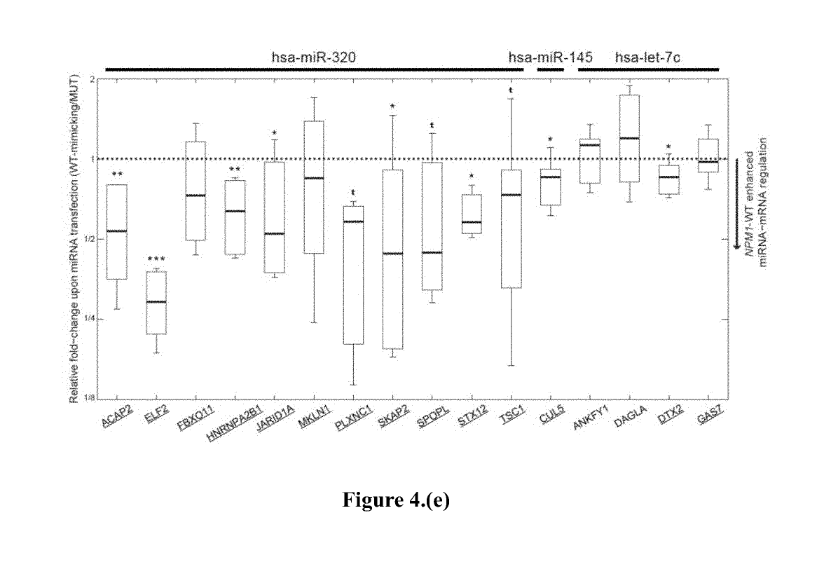

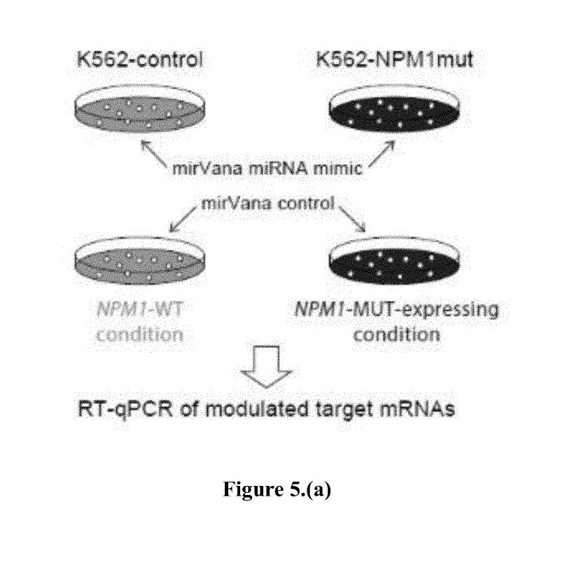

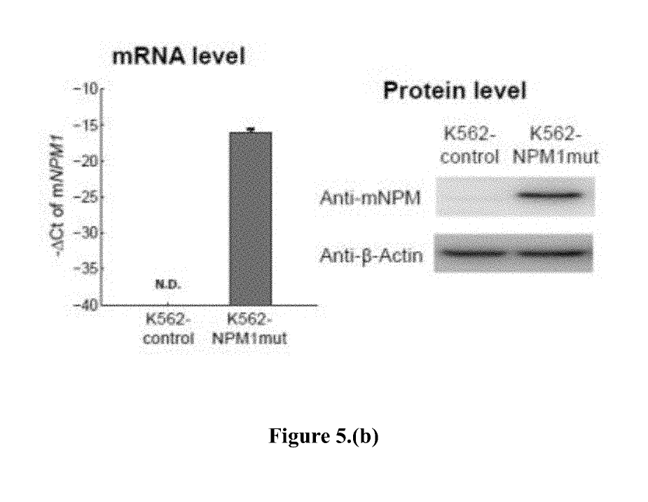

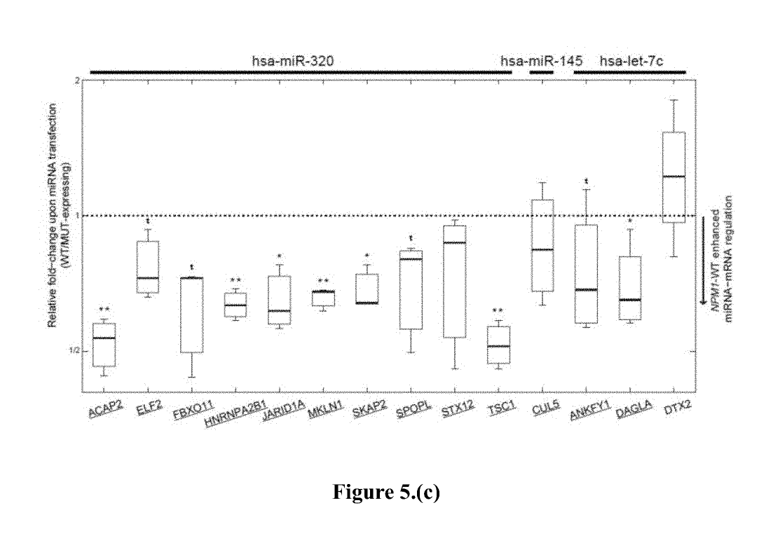

To investigate whether the NPM1 mutation-modulated MMR pairs identified in AML patients shown above were truly modulated by NPM1 mutation per se in AML cells, we employed two in vitro cell models, one using the OCI/AML3 cell line, which harbors an endogenous heterozygous NPM1 mutation, and the other, NPM1-WT K562 cell line. In the first model (FIG. 4a, left panel), knockdown of mutant NPM1, while preserving the wild-type allele, by siRNA (repression efficiency=74.4% and 61.9% for mRNA and protein levels, respectively; FIG. 4b-c) in OCI/AML3 cells (OCI/AML3-shNPM1mut) mimics the NPM1 wild-type-only state. High-throughput microarray profiling of miRNA and mRNA in the OCI/AML3-shNPM1mut and the OCI/AML3-scramble cells (as controls) was used to determine the existence of NPM1 mutation-modulated MMR. Systematic correlation analysis of these paired miRNA and mRNA profiles revealed that the 493 MMR pairs exhibited strengthened negative regulation under NPM1-WTmimicking conditions (OCI/AML3-shNPM1mut), compared to the NPM1-MUT controls (OCI/AML3-scramble) (paired t-test one-tailed P=1.5×10₋₆, and Kolmogorov-Smirnov test P=2.6×10₋₉; FIG. 4d), compatible with the findings in the discovery cohort. Next, to further quantitatively confirm the differential regulation modulated by NPM1 mutation, we electroporated the mimics of hsa-miR-320, hsa-miR-145, or hsa-let-7c into OCI/AML3-shNPM1mut and OCI/AML3-scramble cells, followed by RT-qPCR of their target mRNAs (FIG. 4a, right panel). Among the 16 MMR pairs tested, 14 (87.5%) showed enhanced down-regulation of the target mRNAs upon miRNA transfection into the cells where NPM1-MUT was knocked down (i.e. mimicking the ‘wild-type’ cells), compared to controls (FIG. 4e). The enhancement in regulatory strength in 11 of them (68.8% of the 16 tested MMR pairs) was either strong (statistically significant with one sample t-test P<0.05; 8 pairs) or moderate (P<0.15; 3 pairs). We further tested the modulated MMR using the second cell model, in which mutant NPM1 was overexpressed in the NPM1-WT K562 cell line (K562-NPM1mut cells), with empty vector as control (K562-control cells) (FIG. 5a). Efficiency of the expression construct of NPM1 mutation was confirmed both in mRNA and protein expression (FIG. 5b). Similar to the experiments in OCI/AML3 cells, a majority (11 out of 14, 78.6%) of tested pairs exhibited significantly or a trend (7 and 4 pairs, respectively) of enhanced down-regulation upon miRNA transfection into the cells with wild-type NPM1 (FIG. 5c). Taken together, our data corroborate the existence of modulation effects of NPM1 mutation both in AML cohorts and cell lines.

Survival Significance of NPM1 Mutation-Modulated MMR

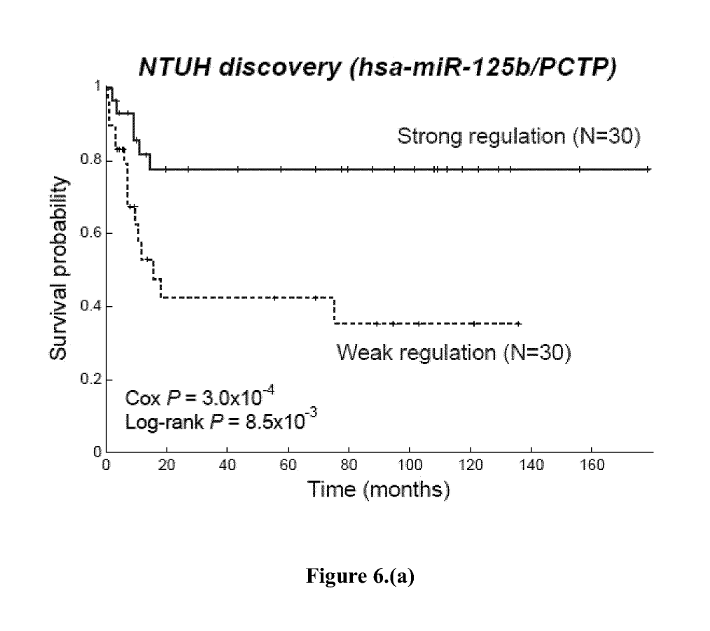

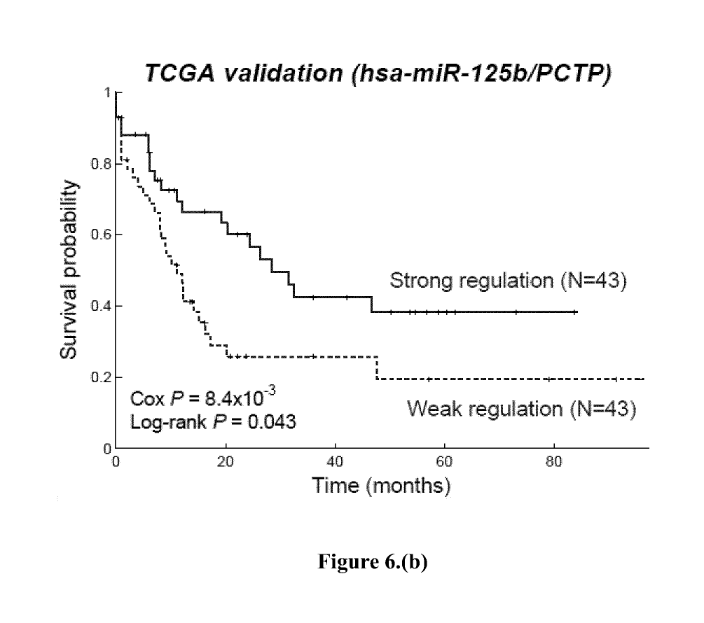

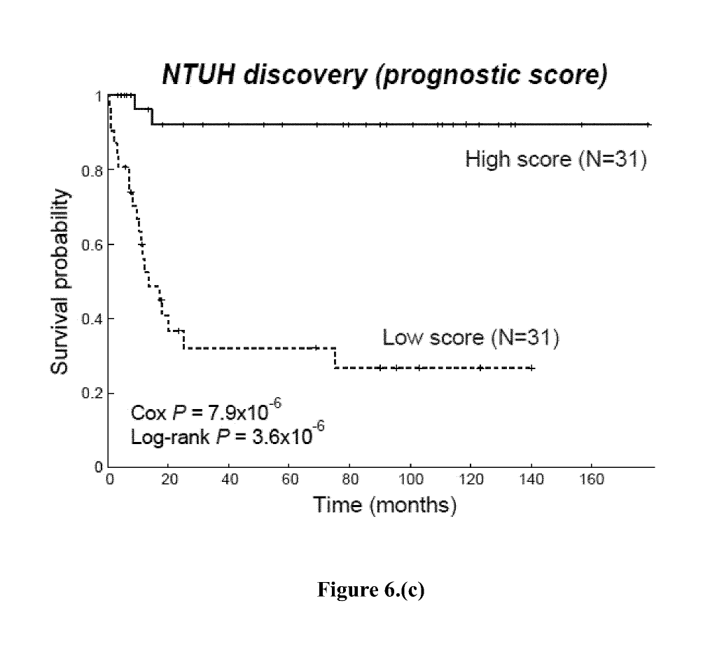

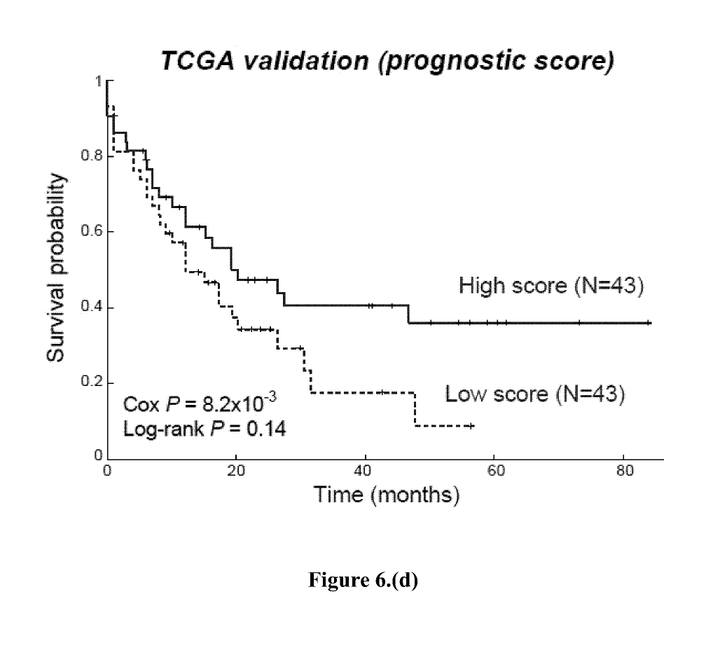

To evaluate the prognostic significance of differential regulation of NPM1 mutation modulated MMR in AML, we conducted Cox proportional hazards regression analysis for OS based on the ‘covariability’ parameter that measures the dynamicity of MMR; i.e., for a given miRNA/mRNA pair, the larger the covariability is, the greater the strength of the MMR in a patient. Patients who received standard intensive chemotherapy₃₁ in the NTUH discovery cohort was analyzed (n=125); patient characteristics were described previously. The most significant prognostic pair was hsa-miR-125b/PCTP (univariable Cox P=3.0×10₋₄; FIG. 6a and Table 1). The favorable implication of strong regulation between hsa-miR-125b and PCTP on OS (median not reached vs. 15.5 months; log-rank P=8.5×10₋₃, FIG. 6a) was validated in the TCGA cohort (n=174; Cox P=8.4×10₋₃ and log-rank P=0.043; median 28.5 vs. 11.2 months; FIG. 6b). The targeting relationship of hsa-miR-125b/PCTP was previously verified in vitro in chronic lymphocytic leukemia (i.e., under the NPM1-WT condition), with implications for inhibition of adaption of cell metabolism to a transformed state.₃₂In addition to hsa-miR-125b/PCTP, we identified eight other prognostic pairs (with univariable Cox P<0.005; Table 1) among the 493 NPM1 mutation-modulated MMR pairs in the discovery dataset. For instance, strong regulatory strength of hsa-miR-125b and ABCC4, a gene related to proliferation/differentiation in leukemia cells and hematopoietic stem cells and acute lymphoblastic leukemia prognosis, was associated with a better OS (Cox P=3.1×10₋₃; Table 1). In another prognostic pair hsa-miR-376c/MEF2C (Cox P=3.0×10₋₃; Table 1), MEF2C is a cooperating oncogene in leukemogenesis. Although these pairs were identified from NPM1 modulation, their prognostic effects were independent of and even out-performed NPM1 mutation and NPM1-MUT/FLT3-ITD-negative status in the NUTH dataset (multivariable Cox P-values of all pairs<0.05; Table 1). In the TCGA cohort, 5 (55.6%) of the nine MMR pairs were independent prognostic factors (P<0.05), and one showed a concordant trend (P<0.15; Table 1). To further corroborate the prognostic significance of MMR, we combined the 9 modulated MMR pairs into a scoring system by a simple weighted sum, where the weights for individual pairs were determined by corresponding β values yielded by the multivariable Cox model taking the 9 pairs as co-variables. The prognostic scoring system was constructed as: Score=0.36×C_{miR-125b/PCTP}+0.25×C_{miR-320/GMCL1}+0.21×C_{miR-193b/KIAA0182}+0.23×C_{miR-193b/ARID4B}+0.24×C_{miR-376c/MEF2C}+0.02×C_{miR-125b/ABCC4}+0.11×C_{miR-320/ANKRD10}+0.03×C_{miR-125b/BIN2}+0.19×C_{miR-125b/ST6GAL1}, where C denotes the covariability of a miRNA and its modulated target mRNA. The prognostic score was predictive of favorable OS in both NTUH and TCGA datasets (univariable Cox P=7.9×10₋₆ and 8.2×10₋₃, respectively; FIG. 6c-d). The score appeared to be a highly independent predictor in both datasets when co-analyzed with several well-known prognostic factors₁₁, including age and white blood cell count at diagnosis, Southwest Oncology Group (SWOG) cytogenetic risk category, NPM1-MUT/FLT3-ITD-negative status, and CEBPA double mutation (Table 2). The prognostic independence remained even when other gene mutations with prognostic significance, including DNMT3A, MLL-PTD, RUNX1, and WT1, were included in the multivariable analysis (Supplementary Table S3); the TCGA dataset was not analyzed since these mutation statuses were not available. Furthermore, the prognostic score was an independent predictor when compared with four prognostic miRNAs or a seven mRNA signature proposed from previous studies. Taken together, our data suggest that the prognostic effects of covariability of MMR pairs are independent of gene mutations and highly consistent, and thus potentially widely applicable in predicting AML prognosis.

NPM1 encodes a multifunctional nucleolar phosphoprotein implied for both tumor suppressor and oncogene functions. Studies have indicated the critical role of NPM1 mutation in leukemogenesis and prognostic prediction in AML patients. Besides the NPM1 mutation associated miRNA and mRNA signatures, a recent study identified handful of novel MMR pairs composed of differentially expressed miRNAs and mRNA with significant inverse changes against these miRNAs in NPM1-MUT and implied a potential role in sensitization to chemotherapy in AML.₈ However, the participation of NPM1 mutation in dynamic miRNA-mRNA interplay, i.e., ‘differential correlation’ or ‘differential regulation’ between miRNAs and mRNAs, remains unexplored. Here we showed NPM1 mutation as a potential modulator of dynamic MMR in AML, which was validated by independent AML cohorts. Compatible with the in vivo findings, in vitro experiments revealed that the NPM1 mutation-modulated MMR pairs identified in AML patients also exhibited a systematically strengthened negative regulation in OCI/AML3 cells under NPM1-WT-like conditions, compared to the NPM1-MUT-expressing controls. In further in vitro validation experiments, hsa-miR-320, a prominent hub (FIG. 2b), and hsa-miR-145 and hsa-let-7c, the miRNAs without significant expressional differences associated with NPM1 mutation (FIG. 2b), were randomly chosen; most of the tested target mRNAs of these three miRNAs showed enhanced down-regulation upon transfection of individual miRNA in the NPM1-WT (or NPM1-WT-mimicking) cells, compared to the NPM1-MUT (or NPM1-MUT-mimicking) cells, indicating the existence of modulation effects of NPM1 mutation on these MMR pairs.

The underlying mechanism of modulation by NPM1 mutation remains to be determined. Only a small portion of the MMR pairs may be directly regulated by NPM1 mutation; i.e., NPM1 mutation-associated differential expression of such miRNAs leads to differential power in regulating their target mRNAs. NPM1 encodes nucleophosmin, a nucleolar protein that shuttles between nucleus and cytoplasm whereas mutated NPM1 gene causes aberrant cytoplasmic dislocation of nucleophosmin. We speculate that NPM1 modulation renders distinct signatures of miRNAs and mRNAs, resulting in disturbed MMR through the competing endogenous RNA (ceRNA) regulation. The concept of ceRNA regulation argues that the regulatory strength of an miRNA-mRNA pair can be modulated by expression of another target mRNA of the miRNA. Indeed, the identified 493 modulated MMR pairs formed slightly more (by ˜8.9%) ceRNA triplets (as defined in a previous report₄₆) with NPM1 mutation-upregulated mRNAs than those unmodulated putative MMR pairs (t-test one-tailed P=5.8×10₋₃); these upregulated mRNAs could act as ‘sponges’ to absorb miRNAs and disturb their associated MMR. This serves as a possible way by which mutated NPM1 interrupts the balance among some miRNAs and their targets. On the other hand, nucleophosmin was shown to bind cooperatively with high affinity for single-stranded nucleic acids with the implication of altering nucleic acid secondary structure. Furthermore, nucleophosmin was suggested to bind miRNAs and implied to play a role in protecting miRNAs against degradation. It is conceivable that the aberrant export of mutated nucleophosmin into the cytoplasm, where miRNA-mRNA targeting takes place, may physically disrupt some miRNA-mRNA pairings, though there is so far no evidence showing differential binding affinity of mutated nucleophosmin to miRNAs or mRNAs. This hypothesis could be tested by CLIP-Seq (cross-linking immunoprecipitation followed by next-generation sequencing) experiments to investigate the binding of wild-type and mutated nucleophosmin, as well as the RNA-induced silencing complex (RISC), on miRNAs or mRNAs. However, as far as we know, there are no antibodies specific to wild-type or mutant nucleophosmin for efficient immunoprecipitation. Future studies to investigate the underlying mechanisms of NPM1 mutation modulation are warranted. The identified modulated MMR was associated with biological functions related to NPM1 mutation and pathways implicated in AML. These findings illustrated that NPM1 mutation may, in addition to regulate expressional signatures of genes, modulate MMR and lead to the development of AML. We also demonstrated the clinical significance of NPM1 mutation-modulated MMR in terms of patient outcome. Specifically, we showed the survival significance of the regulatory strength of nine modulated miRNA/mRNA pairs, with hsa-miR-125b/PCTP as the strongest one. While typical survival prediction models are built on expression levels of biomarkers, dynamic protein-protein interaction was successfully applied to predicting clinical outcome in breast cancer. The present invention is, to our knowledge, the first to show that dynamicity of miRNA-mRNA regulation can predict prognosis in AML patients. For instance, strong regulation between hsa-miR-125b and PCTP was associated with favorable OS. Based on the 9 prognostic pairs, we constructed a prognostic scoring system, in which the regulatory strengths of MMR pairs for each patient were measured by covariability scores. A high score is predictive of favorable OS (FIG. 6c-d). We also showed its high independence and reliability in predicting OS in independent cohorts. However, aside from TCGA, there is, as far as we know, no other publicly available large sample-paired.

TABLE 1
Cox analysis of 9 prognostic NPM1 mutation-modulated miRNA-mRNA regulation pairs for
overall survival in the NTUH discovery and TCGA validation datasets

| | | NTUH discovery | | | | | |
|---|---|---|---|---|---|---|---|
| | | Univariable | | Multivariable | | | |
| | | | HR | | HR | P of | P of NPM1/ |
| miRNA | mRNA | P^{a} | (95% CI)^{a} | P^{a} | (95% CI)^{a} | NPM1^{b} | FLT3-ITD^{c} |
| hsa-miR-125b | PCTP | 3.0E−04 | 0.57 | 4.7E−04 | 0.56 | 2.6E−02 | 0.22 |
| | | | (0.42-0.77) | | (0.41-0.78) | | |
| hsa-miR-320 | GMCL1 | 4.7E−04 | 0.66 | 7.2E−03 | 0.72 | 0.13 | 0.32 |
| | | | (0.52-0.83) | | (0.56-0.91) | | |
| hsa-miR-193b | KIAA0182 | 1.8E−03 | 0.59 | 1.7E−03 | 0.58 | 1.6E−02 | 8.4E−02 |
| | | | (0.42-0.82) | | (0.41-0.82) | | |
| hsa-miR-193b | ARID4B | 2.4E−03 | 0.49 | 4.1E−03 | 0.51 | 0.14 | 0.12 |
| | | | (0.31-0.78) | | (0.32-0.81) | | |
| hsa-miR-376c | MEF2C | 3.0E−03 | 0.56 | 5.4E−03 | 0.57 | 2.1E−02 | 0.20 |
| | | | (0.38-0.82) | | (0.38-0.85) | | |
| hsa-miR-125b | ABCC4 | 3.1E−03 | 0.59 | 5.4E−03 | 0.60 | 3.8E−02 | 0.18 |
| | | | (0.41-0.84) | | (0.42-0.86) | | |
| hsa-miR-320 | ANKRD10 | 3.5E−03 | 0.65 | 2.2E−03 | 0.62 | 7.9E−03 | 4.3E−02 |
| | | | (0.49-0.87) | | (0.46-0.84) | | |
| hsa-miR-125b | BIN2 | 4.3E−03 | 0.59 | 8.7E−03 | 0.60 | 4.3E−02 | 0.26 |
| | | | (0.41-0.85) | | (0.41-0.88) | | |
| hsa-miR-125b | ST6GAL1 | 4.8E−03 | 0.49 | 6.2E−03 | 0.47 | 2.3E−02 | 0.22 |
| | | | (0.29-0.80) | | (0.27-0.81) | | |

| | TCGA validation | | | | | |
|---|---|---|---|---|---|---|
| | Univariable | | Multivariable | | | |
| | | HR | | HR | P of | P of NPM1/ |
| miRNA | P^{a} | (95% CI)^{a} | P^{a} | (95% CI)^{a} | NPM1^{b} | FLT3-ITD^{c} |
| hsa-miR-125b | 8.4E−03 | 0.66 | 5.9E−03 | 0.64 | 0.64 | 0.71 |
| | | (0.48-0.90) | | (0.46-0.88) | | |
| hsa-miR-320 | 0.96 | 0.99 | 0.92 | 1.01 | 0.44 | 0.72 |
| | | (0.82-1.21) | | (0.83-1.22) | | |
| hsa-miR-193b | 0.84 | 1.02 | 0.83 | 1.02 | 0.44 | 0.71 |
| | | (0.85-1.22) | | (0.85-1.23) | | |
| hsa-miR-193b | 0.14 | 0.84 | 0.11 | 0.82 | 0.54 | 0.64 |
| | | (0.67-1.06) | | (0.64-1.05) | | |
| hsa-miR-376c | 4.5E−02 | 0.78 | 4.8E−02 | 0.79 | 0.74 | 0.70 |
| | | (0.62-0.99) | | (0.62-1.00) | | |
| hsa-miR-125b | 1.7E−02 | 0.73 | 7.2E−03 | 0.68 | 0.55 | 0.63 |
| | | (0.56-0.95) | | (0.52-0.90) | | |
| hsa-miR-320 | 0.65 | 0.95 | 0.71 | 0.96 | 0.47 | 0.74 |
| | | (0.76-1.18) | | (0.78-1.19) | | |
| hsa-miR-125b | 1.0E−02 | 0.68 | 7.6E−03 | 0.67 | 0.74 | 0.88 |
| | | (0.51-0.91) | | (0.50-0.90) | | |
| hsa-miR-125b | 1.1E−02 | 0.60 | 8.6E−03 | 0.56 | 0.84 | 0.82 |
| | | (0.40-0.89) | | (0.36-0.86) | | |

Abbreviations:
P, Cox P-value;
HR, hazard ratio;
CI, confidence interval.
^{a}Results of miRNA-mRNA covariability.
^{b}NPM1-MUT vs. NPM1-WT.
^{c}NPM1-MUT/FLT3-ITD-negative vs. other subtypes.

TABLE 2
Cox analysis of the prognostic score for overall survival in
the NTUH discovery and TCGA validation datasets

| | NTUH discovery | | | TCGA validation | | |
|---|---|---|---|---|---|---|
| | | Hazard | 95% confidence | | Hazard | 95% confidence |
| Variable | P-value | ratio | interval | P-value | ratio | interval |
| Age^{a} | 7.3E−04 | 2.91 | 1.57-5.41 | 2.0E−03 | 2.06 | 1.30-3.27 |
| WBC^{b} | 0.10 | 1.65 | 0.90-3.00 | 0.87 | 0.96 | 0.61-1.51 |
| Karyotype^{c} | 0.27 | 1.60 | 0.69-3.69 | 0.38 | 1.23 | 0.77-1.97 |
| NPM1/FLT3-ITD^{d} | 0.13 | 0.50 | 0.20-1.22 | 0.90 | 1.04 | 0.58-1.85 |
| CEBPA^{e} | 5.4E−02 | 0.14 | 0.02-1.04 | — | — | — |
| Score | 4.5E−05 | 0.46 | 0.32-0.67 | 3.6E−02 | 0.76 | 0.59-0.98 |

^{a}Age older than 50 years vs. 50 years or younger.
^{b}White blood cell >50,000/μL vs. ≤50,000/μL.
^{c}Southwest Oncology Group (SWOG) cytogenetic risk categories: unfavorable cytogenetics vs others.
^{d}NPM1-MUT/FLT3-ITD-negative vs. others.
^{e}CEBPA double mutation vs. others.

Although specific embodiments have been illustrated and described, it will be obvious to those skilled in the art that various modifications may be made without departing from what is intended to be limited solely by the appended claims.

What is claimed is:

1. A method for predicting prognostic results of diseases including acute myeloid leukemia, said method comprising: analyzing regulatory strengths of a NPM1 mutation-modulated miRNA/mRNA regulation (MMR) pair in a subject.

2. The method of claim 1, wherein the NPM1 mutation-modulated miRNA/mRNA regulation (MMR) pair comprises hsa-miR-125b, hsa-miR-193b, hsa-miR-320 and hsa-miR-376c.

3. The method of claim 1, wherein the NPM1 mutation-modulated miRNA/mRNA regulation (MMR) pair comprises PCTP, GMCL1, KIAA0182, ARID4B, MEF2C, ABCC4, ANKRD10, BIN2 and ST6GAL1.

4. The method of claim 1, wherein the NPM1 mutation-modulated miRNA/mRNA regulation (MMR) pair is hsa-miR-125b/PCTP.

5. The method of claim 1, wherein the NPM1 mutation-modulated miRNA/mRNA regulation (MMR) pair is hsa-miR-125b/ABCC4.

6. The method of claim 1, wherein the NPM1 mutation-modulated miRNA/mRNA regulation (MMR) pair is hsa-miR-125b/BIN2.

7. The method of claim 1, wherein the NPM1 mutation-modulated miRNA/mRNA regulation (MMR) pair is hsa-miR-125b/ST6GAL1.

8. The method of claim 1, wherein the NPM1 mutation-modulated miRNA/mRNA regulation (MMR) pair is hsa-miR-193b/KIAA0182.

9. The method of claim 1, wherein the NPM1 mutation-modulated miRNA/mRNA regulation (MMR) pair is hsa-miR-193b/ARID4B.

10. The method of claim 1, wherein the NPM1 mutation-modulated miRNA/mRNA regulation (MMR) pair is hsa-miR-320/GMCL1.

11. The method of claim 1, wherein the NPM1 mutation-modulated miRNA/mRNA regulation (MMR) pair is hsa-miR-320/ANKRD10.

12. The method of claim 1, wherein the NPM1 mutation-modulated miRNA/mRNA regulation (MMR) pair is hsa-miR-376c/MEF2C.

13. The method of claim 1, wherein the subject comprises cell samples, tissue sections, blood samples and lymph samples.

14. The method of claim 13, wherein the cell lines comprise OCI/AML3 and NPM1-WT K562.

15. A prognostic scoring system, said scoring system being the following equation:
Score=0.36×C_{miR-125b/PCTP}+0.25×C_{miR-320/GMCL1}+0.21×C_{miR-193b/KIAA0182}+0.23×C_{miR-193b/ARID4B}+0.24×C_{miR-376c/MEF2C}+0.02×C_{miR-125b/ABCC4}+0.11×C_{miR-320/ANKRD10}+0.03×C_{miR-125b/BIN2}+0.19×C_{miR-125b/ST6GAL1},
where C denotes the covariability of a miRNA and its modulated target mRNA.

16. The prognostic scoring system of claim 15, being applied to predict acute myeloid leukemia (AML) prognosis.
